(19) United States
(12) Patent Application Publication
(10) Pub. No.: US 2020/0237203 A1
(43) Pub. Date: Jul. 30, 2020

(54) LUMENED DEVICE FLUSHING ASSEMBLY AND METHOD OF USE

(71) Applicant: 3M INNOVATIVE PROPERTIES COMPANY, SAINT PAUL, MN (US)

(72) Inventors: GUENTER M. ZILLIGEN, GREVENBROICH (DE); HENNING REUTER, WILLICH (DE); KARL-HEINZ RECHLITZ, WUPPERTAL (DE); MANUEL STEINBRINK, KORSCHENBROICH (DE); G. MARCO BOMMARITO, STILLWATER, MN (US)

(73) Assignee: 3M INNOVATIVE PROPERTIES COMPANY, SAINT PAUL, MN (US)

(21) Appl. No.: 16/652,997

(22) PCT Filed: Oct. 2, 2018
(86) PCT No.: PCT/IB2018/057653

§ 371 Date: Apr. 2, 2020

Related U.S. Application Data
(60) Provisional application No. 62/567,565, filed on Oct. 3, 2017.

(51) Int. Cl. A61B 1/12; A61B 1/00; A61L 2/28

(52) U.S. Cl. CPC A61B 1/125; A61B 1/00137; A61L 2202/24; A61L 2/28; A61B 1/00142

(57) ABSTRACT

An endoscope flushing assembly is provided. In some embodiments, the flushing assembly may include an adapter and a stand. In some embodiments, the flushing assembly may further comprise a stopper. In some embodiments, the flushing assembly may further comprise a sample fluid tube. In some embodiments, the flushing assembly may further comprise a fluid receptacle. Also provided are a flushing assembly kit and a method of using the endoscope flushing assembly to evaluate the cleanliness and/or disinfection of an endoscope.

### TECHNICAL FIELD

This disclosure relates in general to the field of evaluation of lumened devices and more particularly to an assembly and method for assessing the cleanliness and/or disinfection of an endoscope.

### BACKGROUND

Millions of gastrointestinal endoscopy procedures are performed on an annual basis around the world. These endoscopy procedures are typically performed using complex, flexible instruments that, when inserted into a patient's body, may become contaminated with biomaterial and microorganisms, including potential pathogens. Thus, careful and thorough cleaning of endoscopes between patients is critical to reducing the risk of cross-contamination and the possible transmission of pathogens during an endoscopy procedure.

The cleaning of endoscope channels, i.e., lumens, can be particularly demanding, as they may be convoluted and too narrow to accommodate a scrub brush. As a result, pathogens may adhere to the interior surfaces of the endoscope even after a thorough cleaning Because endoscope channels do not lend themselves to visual inspection and harsh sterilization procedures may be unsuitable for some endoscopes, it is therefore advantageous to be able to monitor the cleanliness and/or disinfection of an endoscope with a procedure that is simple, accurate, and reduces the risk of cross-contamination of medical instruments.

### SUMMARY

In one aspect, provided is an endoscope flushing assembly 100 comprising an adapter 200, where the adapter 200 is configured to support a distal portion of an endoscope and receive a sample fluid from the distal portion of the endoscope. In some embodiments, the adapter 200 comprises a fluid guide 250. In some embodiments, the flushing assembly 100 further comprises a stand 300 configured to support the adapter 200. In some embodiments, the stand 300 comprises a notch 350 configured to secure the adapter 200. In some embodiments, the endoscope flushing assembly 100 further comprising a stopper 400. In some embodiments, the stopper 400 comprises a first port plug 420 and a second port plug 440. In some embodiments, the endoscope flushing assembly 100 further comprises a tube 500, wherein the tube 500 is configured to releasably connect to a fluid inlet of the endoscope. In some embodiments, the tube 500 comprises a silicone rubber. In some embodiments, the endoscope flushing assembly 100 further comprises a fluid receptacle 600. method comprising sealing an open endoscope port with a stopper 400, securing an adapter 200 to a stand 300, inserting the distal end of the endoscope into the adapter 200, connecting a first end of a tube 500 to a fluid inlet of the endoscope and a second end of the tube 500 to a fluid source comprising a sample fluid, flushing the endoscope interior with the sample fluid, collecting the sample fluid at an outlet 220 of the adapter 200, and analyzing the sample fluid for the presence of a contaminant.

Features and advantages of the present disclosure will be further understood upon consideration of the detailed description as well as the appended claims.

### BRIEF DESCRIPTION OF THE DRAWINGS

FIG. 1 is a perspective view showing engagement of an endoscope with an embodiment of a flushing assembly of the present disclosure.

FIG. 2 is a front perspective view of an embodiment of an adapter of the present disclosure.

FIG. 3 is a back perspective view of an embodiment of an adapter of the present disclosure.

FIG. 4 is a side sectional view of an embodiment of an adapter of the present disclosure.

FIG. 5 s a back sectional view of an embodiment of an adapter of the present disclosure.

FIG. 6 is a side sectional view of an embodiment of an adapter of the present disclosure.

FIG. 7 is a front perspective view of an embodiment of a stand of the present disclosure.

FIG. 8 is a side sectional view of an embodiment of a stand of the present disclosure.

FIG. 9 is a front elevational view of an embodiment of a stand of the present disclosure.

FIG. 10 is a front perspective view showing engagement of an embodiment of an adapter, a stand, and a fluid receptacle of the present disclosure.

FIG. 11 is a front perspective view showing engagement of the distal end of an endoscope with the distal end of an embodiment of an adapter of the present disclosure.

FIG. 12 is a perspective view an embodiment of a stopper of the present disclosure.

FIG. 13 is a perspective view showing engagement of an embodiment of a stopper with endoscope ports according to the present disclosure.

FIG. 14 is a perspective view of engagement of an endoscope port with an embodiment of a tube of the present disclosure.

FIG. 15 is a perspective view of an embodiment of an adapter of the present disclosure.

FIG. 16 is a perspective view of the adapter of FIG. 15 engaging with an embodiment of a stand of the present disclosure.

FIG. 17 is a perspective view of an embodiment of an adapter and a fluid receptacle of the present disclosure.

[symbol]

engagement of the adapter and fluid receptacle with an embodiment of a stand of the present disclosure.

FIG. 19 is a perspective view of an embodiment of an adapter of the present disclosure.

FIG. 20 is a perspective view of the adapter of FIG. 19 engaging with an embodiment of a stand of the present disclosure.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

Flushing a lumened device, such as, for example, an endoscope with sample fluid, e.g., sterile water, saline, special recovery fluids, such as broth, and collection of the fluid sample as it exits the endoscope can provide the endoscope operator with the opportunity to test the fluid sample for residual contaminants e.g., ATP, protein, microbial contamination, that may remain in the endoscope interior after reprocessing, such as cleaning and/or disinfection, thus providing a measure for cleanliness and/or disinfection and efficacy of endoscope reprocessing methods and operations.

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the term “coupled” and variations thereof are used broadly and encompass both direct and indirect couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

Provided in the present disclosure is an assembly for flushing a lumened device, such as an endoscope, with a sample fluid.

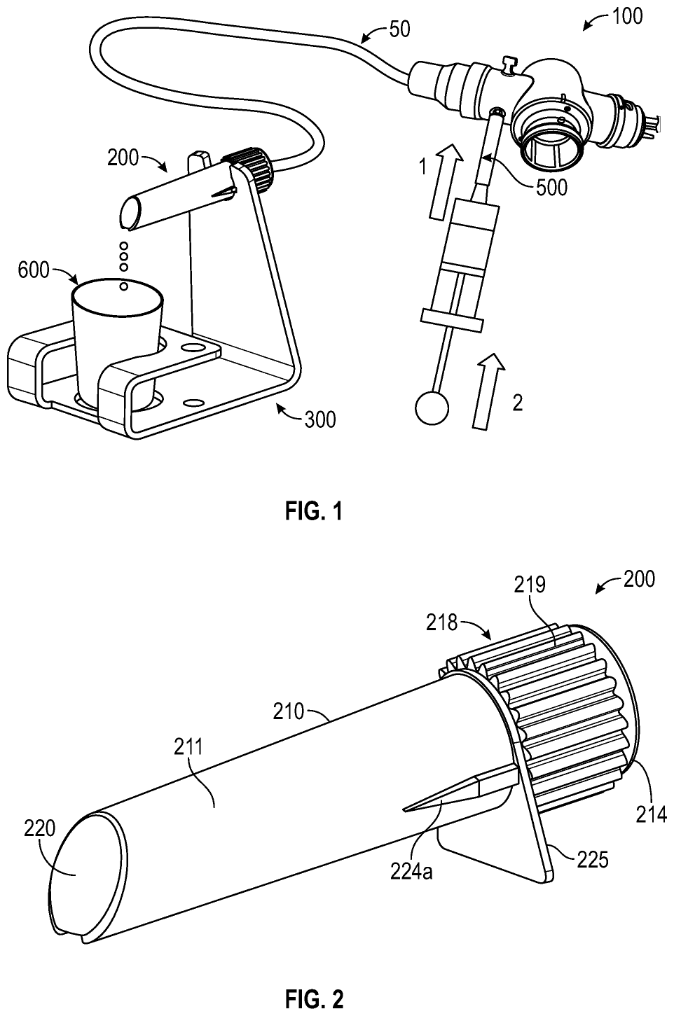

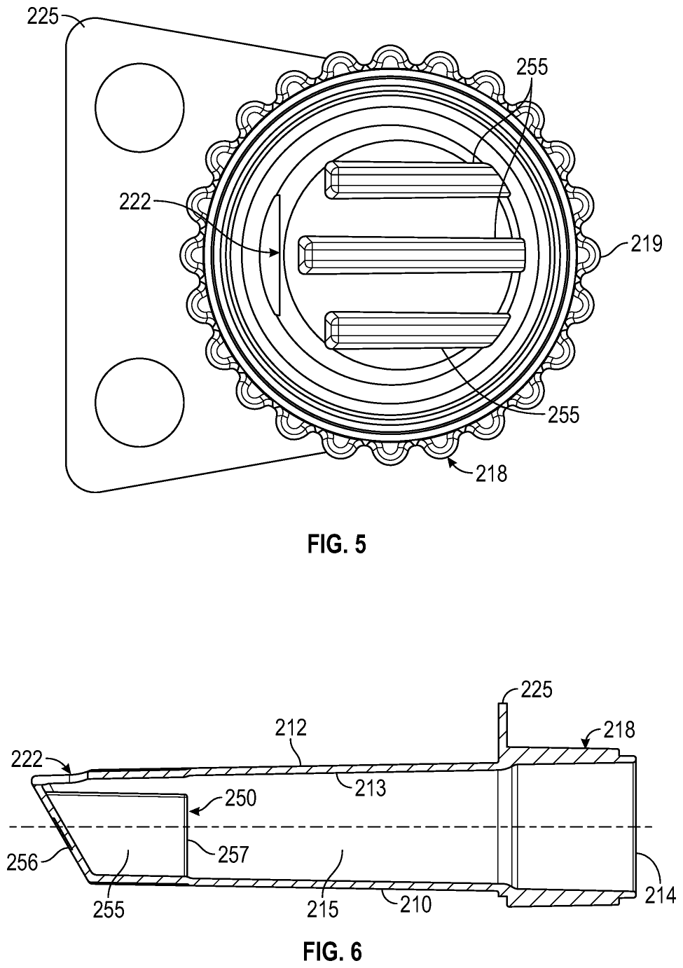

FIGS. 1-14 illustrate an endoscope flushing assembly 100 according to one embodiment of the present disclosure. FIG. 1 illustrates an assembly 100 coupled to an endoscope 50. As shown in FIG. 1, the assembly 100 can include an adapter 200, a stand 300, a stopper 400 (not shown), and a sample fluid tube 500.

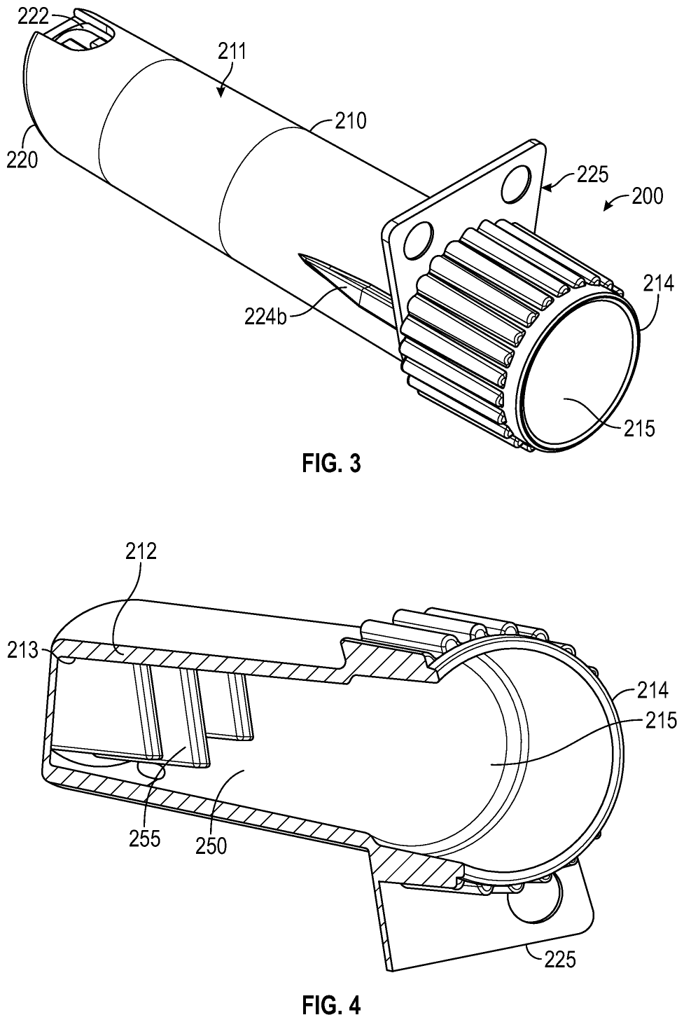

As shown in FIGS. 2 and 3, the adapter 200 may include a housing 210. In some embodiments, and as shown in FIGS. 2 and 3, the housing 210 may be generally frustoconical, but other geometries, e.g., cylindrical, rectangular, are possible for the housing 210. The housing 210 can include at least one liquid-impermeable wall 211 having an outer surface 212 and an inner surface 213 opposite the outer surface 212, a first end 214 configured to receive a distal portion of an endoscope in a reservoir 215, and a second end 220 opposite the first end 214. Commonly, the first end 214 and the reservoir 215 are configured to accommodate an endoscope having a distal end diameter of 5 mm to 16 mm, though the first end 214 and the reservoir 215 may be configured to accommodate an endoscope having a distal end diameter outside of that range, i.e., less than 5 mm or greater than 16 mm. The second end 220 can include aperture 222 which may allow the sample fluid to exit the adapter 200. In some embodiments, and as shown in FIGS. 2 and 3, the aperture 220 may have a semicircular geometry, but other geometries, e.g., rectangular, circular, triangular, are contemplated.

The housing 210 may include a grip area 218 proximal to the first end 214. The grip area 218 may include ribbbing 219 as shown in FIGS. 2 and 3, but also may include a projection of a different geometry, e.g., a hemisphere, a cylinder, which may enhance the ability of an operator to securely hold the adapter 200. The housing 210 may further include one or more protrusions 224a, 224b configured to engage with a complimentary groove 314 of a holder 300 (described below). As shown in FIGS. 2 and 3, the one or more protrusions 224a, 224b may have a generally conical geometry, but other geometries, e.g., trapezoidal, pyramidal, may be used. In some embodiments, the housing 210 may further include a stop 225 proximal to the grip area 218. The stop 225, as shown in FIGS. 2 and 3, may be generally trapezoidal in shape, but other shapes, e.g., rectangular, triangular, semicircular, are contemplated.

Referring to FIGS. 4-6, a fluid guide 250 may extend from the housing 210 wall 211 inner surface 213 into the reservoir 215, proximal to the second end 220, and generally over the aperture 222. The fluid guide 250 may include a front edge 256 and a back edge 257. The fluid guide 250 serves at least two purposes in the adapter 200. First, it can prevent the proximal end of the endoscope from covering the aperture 222 and second, it may reduce the tendency of the sample fluid to splatter or splash as it exits the adapter 200 through the aperture 222.

In some embodiments, and as shown in FIGS. 4-6, the fluid guide 250 may include one or more fins 255 having a generally trapezoidal shape and oriented as shown, though other shapes, e.g., fins 255 are possible. In some embodiments, the fluid guide 250 may comprise a mesh or sieve. In some embodiments, the fluid guide 250 may include three fins 255 spaced 3 mm to 4 mm apart, e.g., 3.4 mm, apart and no more than 4 mm from the inner surface 213 of the wall 211. In some embodiments, the fluid guide 250 can keep the proximal end of the endoscope at least 15 mm to 25 mm, e.g., 17.4 mm, from the second end 220 of the housing 210 when the endoscope is positioned in the reservoir 215. In some embodiments, the fluid guide 250 back edge 257 may be 75 mm to 85 mm, e.g., 80 mm, from the first end 214 of the housing 210.

An adapter 200 of the present disclosure may be fabricated according to methods known to those of ordinary skill in the relevant arts such as, for example, molding, pressing, casting, additive manufacturing (e.g., three-dimensional printing, generic manufacturing), subtractive manufacturing (e.g., machining, turning, milling, drilling), and combinations thereof. In some embodiments, the adapter 200 may be fabricated as one, unitary piece. In some embodiments, components of the adapter 200, such as, for example, the housing 210 and the fluid guide 250, may be fabricated separately and then joined together by a suitable means such as, for example, heating, gluing, soldering, welding, press fitting, cone fitting, snap fitting, sealing, and combinations thereof. Suitable materials for the adapter 200 can include, but are not limited to, a glass, a metal, (e.g., foil), a polymer (e.g., polypropylene, polycarbonate, polyphenylene, polythyene, polystyrene, polyester (e.g., polyethylene terephthalate), polymethyl methacrylate (PMMA or acrylic), acrylonitrile butadiene styrene, cyclo olefin polymer, cyclo olefin copolymer, polysulfone, polyethersulfone, polyetherimide, polybutyleneterephthalate), a ceramic, a porcelain, or combinations thereof In some embodiments, the adapter 200 is transparent or translucent. In some embodiments, the adapter comprises polypropylene.

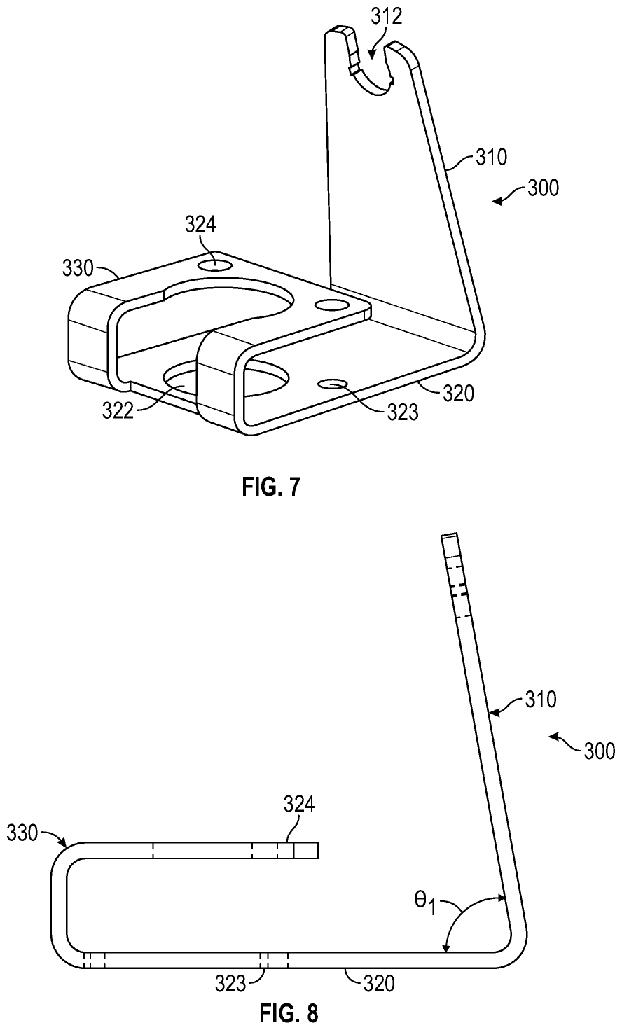

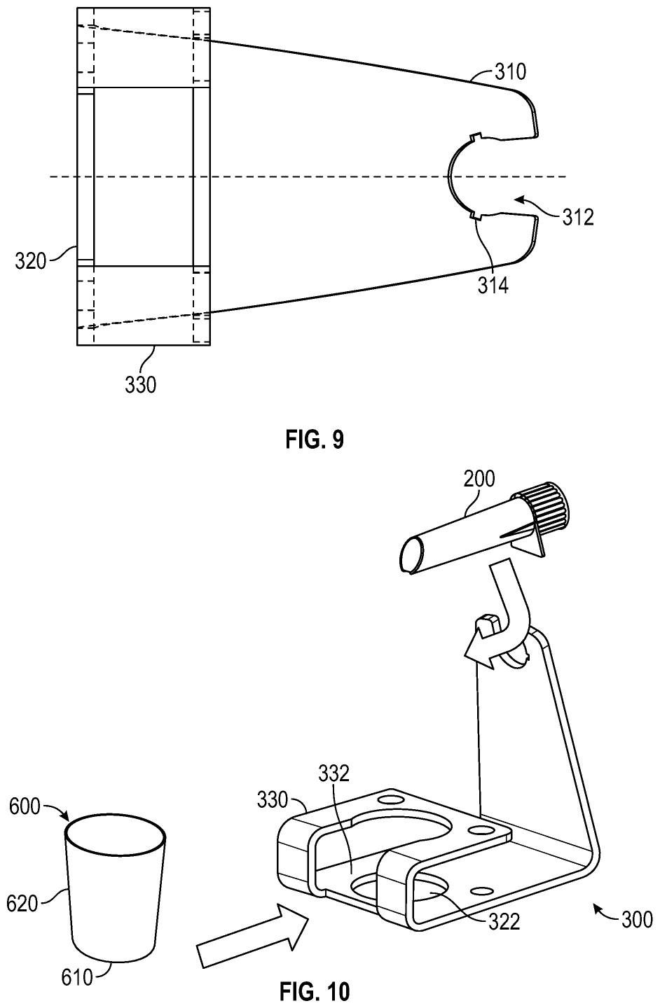

The stand 300 is shown in FIGS. 7-9. Referring to FIG. 7, the stand 300 comprises a support plate 310, a base 320, and a fluid receptacle holder 330. The support plate 310 can include a notch 312 configured to support the adapter 200. In some embodiments, and as shown in FIG. 9, the notch 312 may comprise a groove 314 having a shape that is complimentary to one or more protrusions 224a, 224b on the adapter 200. The groove 314 can provide additional securement of the adapter 200 to the stand 300 in addition to providing visual and tactile signals to the operator regarding the proper orientation of the adapter 200 in the stand 300.

As shown in FIG. 8, the support plate 310 and the base 320 form an angle θ₁ that in some embodiments is less than 90° and greater than 65°, 85° to 75°, 83° to 77°, or 81° to 79°. In some embodiments, angle θ₁ is 80°. The angle θ₁ is significant, as it determines the angle of the adapter 200 relative to the surface on which the stand 300 is resting. Positioning the adapter 200 such that the first end 214 is higher than the second end 220 facilitates flow of a sample solution out of the aperture 222 and allows gravity to assist in maintaining the desired position of the distal end of the endoscope of the fluid guide 250.

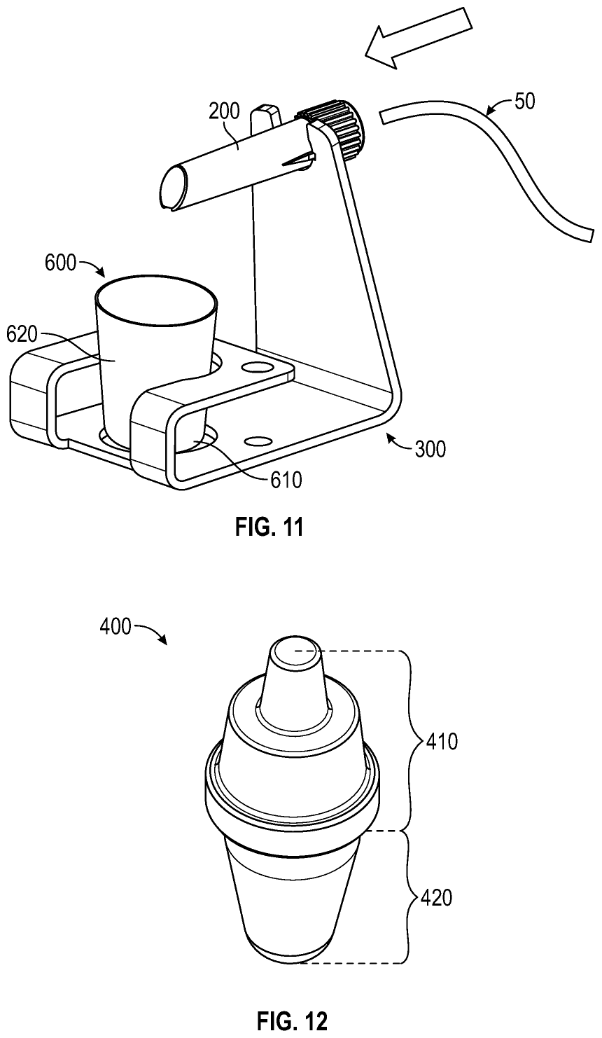

As shown in FIG. 7, the base 320 may include a first hole 322 dimensioned to accommodate, for example, the bottom 610 of a fluid receptacle 600 (shown in FIGS. 10 and 11). In some embodiments, the base 320 may further comprise a second hole 323 dimensioned to accommodate the end of a device such as, for example, a CLEAN-TRACE ATP test device, commercially available from 3M Company, Minnesota, USA.

The fluid receptacle holder 330, as shown in FIG. 8, can have an “L-shaped” configuration and may include an opening 332 configured to accommodate a central portion 620 of a fluid receptacle 600 (shown in FIG. 11). In some embodiments, fluid receptacle holder 330 may further comprise a hole 324 aligned with hole 323, and dimensioned to accommodate a central portion of a device such as, for example, a CLEAN-TRACE ATP test device, commercially available from 3M Company, Minnesota, USA.

A stand 300 of the present disclosure may be fabricated according to methods know to those of ordinary skill in the relevant arts such as, for example, molding, pressing, casting, additive manufacturing (e.g., three-dimensional printing, generic manufacturing), subtractive manufacturing (e.g., machining, turning, milling, drilling), and combinations thereof. In some embodiments, the stand 300 may be fabricated as one, unitary piece. In some embodiments, components of the stand 300, such as, for example, the support plate 310, the base 320, and the fluid receptacle holder 330, may be fabricated separately and then joined together by a suitable means such as, for example, heating, gluing, soldering, welding, press fitting, cone fitting, snap fitting, sealing, and combinations thereof. Suitable materials for the stand 300 can include, but are not limited to, a glass, a metal, (e.g., aluminum), a polymer (e.g., polypropylene, polyethylene, polycarbonate, and other resins inert to sample fluid) a ceramic, a porcelain, and combinations thereof.

The stand 300 may have a weight sufficient to hold the adapter 200 in place after it has been coupled to an endoscope and during the flushing procedure. In some embodiments, the stand 300 has a weight of at least about 0.5 kg, at least about 1.0 kg, at least about 1.5 kg, or at least about 2.0 kg. In some embodiments, it may be desirable to secure the stand to a surface with, for example, a magnetic mount, a suction cup, a clamp, a bolt and/or screw through the base, a bayonet, a hook and loop fastener, adhesive tape, and combinations thereof.

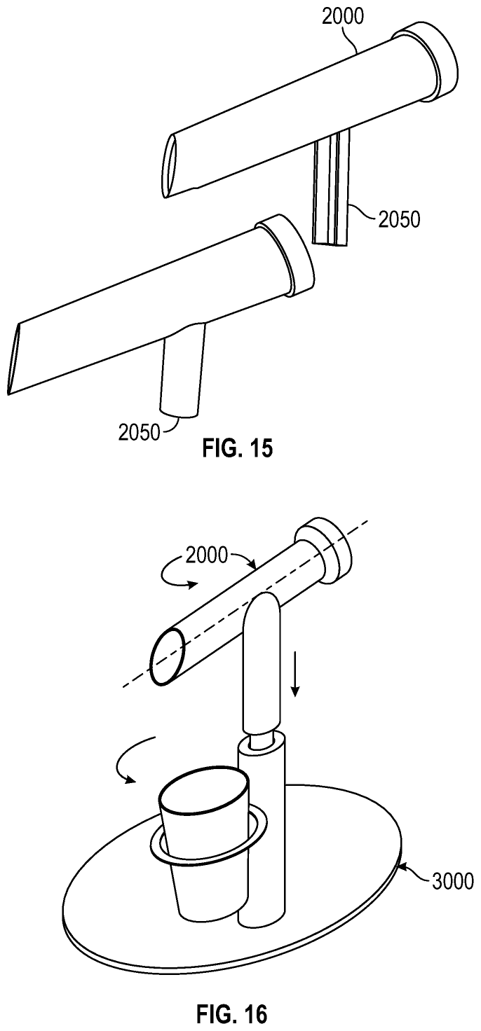

A second embodiment of an adapter 2000 and a stand 3000 is shown in FIGS. 15 and 16. Referring to FIGS. 15 and 16, adapter 2000 may include a post 2050 configured to engage with a stand 3000.

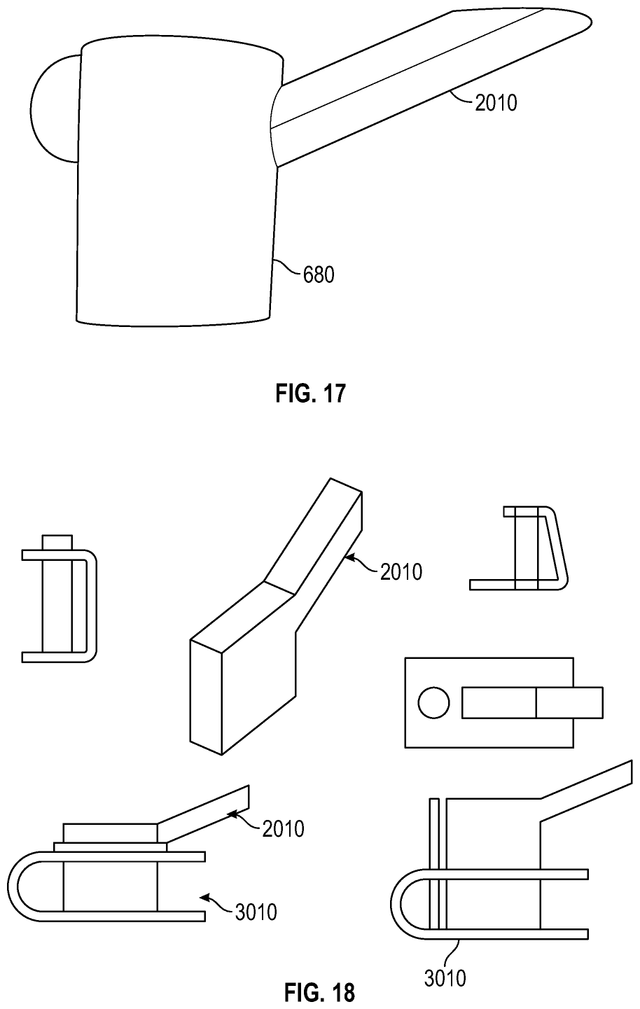

Referring to FIGS. 17 and 18, adapter 2010 may include an integrated fluid receptacle 680 configured to engage with a stand 3010.

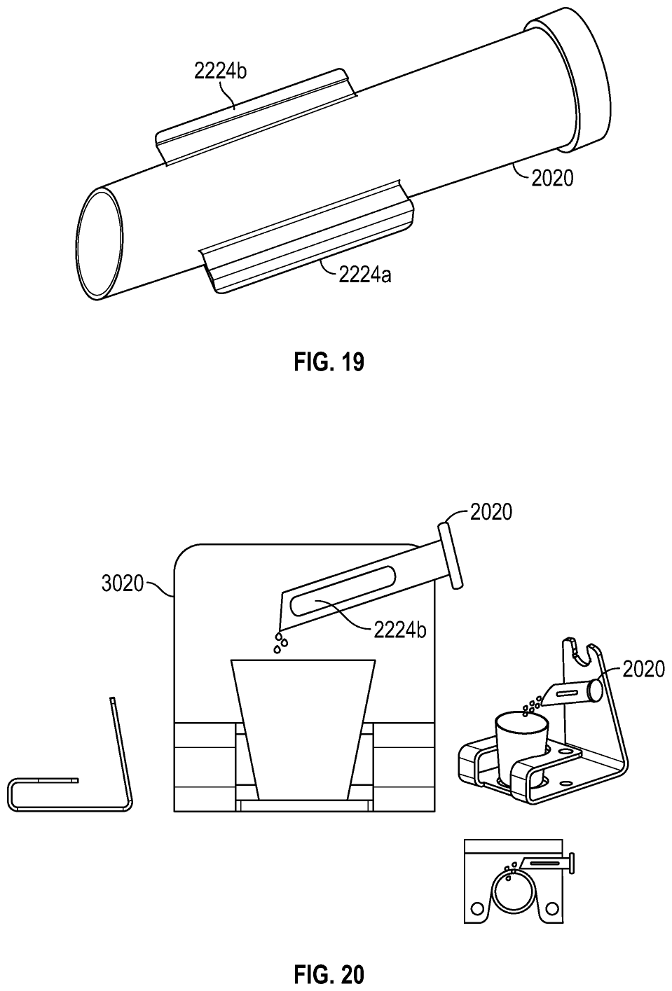

A fourth embodiment of an adapter 2020 and a stand 3020 is shown in FIGS. 19 and 20. Referring to FIGS. 19 and 20, adapter 2020 may include one or more protrusions 2224a, 2224b configured to engage with a stand 3020.

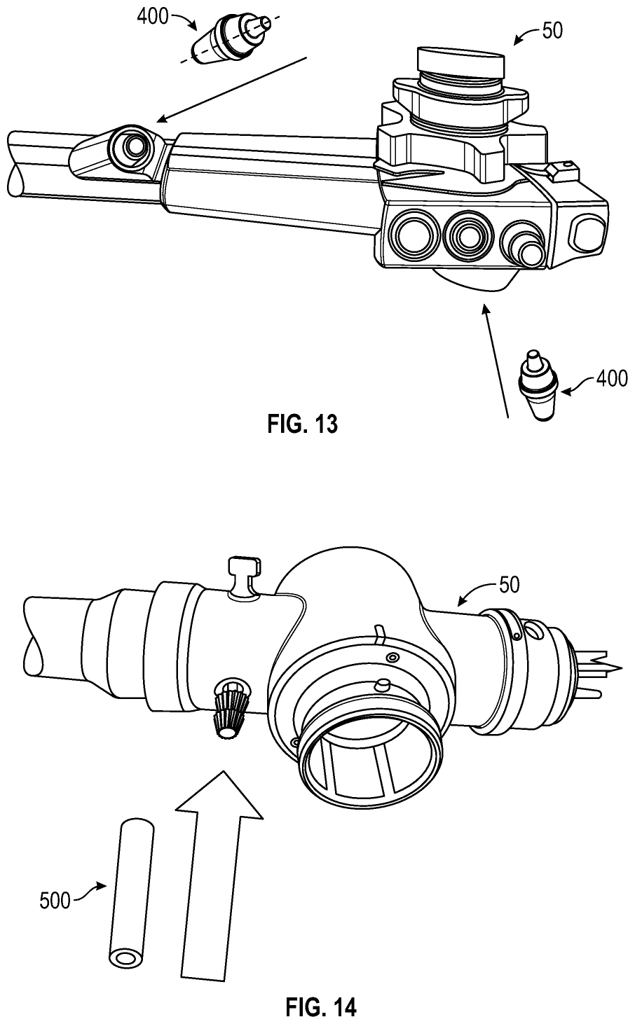

A stopper 400 suitable for use in an assembly 100 of the present disclosure is shown in FIG. 12, and is described in U.S. Patent Application 62/567,522, filed Oct. 3, 2017. FIG. 13 illustrates locations on an endoscope that could be reversibly sealed with the stopper 400 comprising a first port plug 410 and a second port plug 420.

A sample fluid tube 500 suitable for use in an assembly 100 of the present disclosure is shown in FIG. 14. The sample fluid tube 500 may comprise a hollow tube made of a suitable rubber material, e.g., a silicone rubber, having a diameter compatible on a first end with an endoscope inlet port and on a second end with a fluid source, e.g., a syringe, a fluid pump, a valved reservoir.

A fluid receptacle 600 suitable for use in an assembly 100 of the present disclosure is shown in FIGS. 10 and 11. As shown in FIGS. 10 and 11, the fluid receptacle 600 may have a bottom 610 and a central portion 620 configured to releasably engage with the stand 300. Suitable materials for the fluid receptacle 600 can include, but are not limited to, a glass, a metal, (e.g., aluminum), a polymer (e.g., polypropylene, polyethylene, polycarbonate, and other resins inert to sample fluid) a ceramic, a porcelain, or combinations thereof.

Components of an endoscope flushing assembly 100 of the present disclosure may be provided in the form of a kit. In some embodiments, the kit may include an adapter 200 and a stand 300. In some embodiments, the kit may further comprise a stopper 400. In some embodiments, the kit may further comprise a sample fluid tube 500. In some embodiments, the kit may further comprise a fluid receptacle 600. In some embodiments, the kit may further comprise a fluid source, e.g., a syringe. The kit may be provided in a suitable container such as, for example, a box (e.g., metal, paper, plastic), a bag (e.g., paper, plastic), and combinations thereof.

### Method of Flushing a Lumened Assembly

An endoscope flushing assembly 100 of the present disclosure may be used for flushing a lumened device, such as an endoscope, with a sample fluid to evaluate the cleanliness and/or disinfection of an endoscope. The first steps of this process, which may be executed in any convenient order, include: sealing an open endoscope port with a stopper 400, securing an adapter 200 to a stand 300 by, for example, sliding the adapter 200 into the notch 312 in the support plate 310 from the back to the front of the stand 300, as shown in FIG. 10, or mounting the adapter 200 into the adapter 200, and connecting a first end of a tube 500 to a fluid inlet of the endoscope and a second end of the tube 500 to a fluid source comprising a sample fluid. After completion of these first steps, the endoscope interior may be flushed with the sample fluid by activating the fluid source, e.g., depressing the syringe plunger, followed by collecting the sample fluid at an outlet 220 of the adapter 200. The collected sample fluid may then be analyzed for the presence of a contaminant, using, for example, a CLEAN TRACE Clinical ATP system, commercially available from 3M Company, Minnesota, USA. Depending on the results of the fluid analysis, the lumened device may either proceed to clinical use or may be sent for further reprocessing.

1. An endoscope flushing assembly 100 comprising:
an adapter 200, wherein the adapter 200 is configured to support a distal portion of an endoscope and receive a sample fluid from the distal portion of the endoscope.

2. The endoscope flushing assembly 100 of claim 1, wherein the adapter 200 comprises a fluid guide 250.

3. The endoscope flushing assembly 100 of claim 1, further comprising a stand 300 configured to support the adapter 200.

4. The endoscope flushing assembly 100 of claim 3, wherein the stand 300 comprises a notch 350 configured to secure the adapter 200.

5. The endoscope flushing assembly 100 of claim 1, further comprising a stopper 400.

6. The endoscope flushing assembly 100 of claim 5, wherein the stopper 400 comprises a first port plug 420 and a second port plug 440

7. The endoscope flushing assembly 100 of claim 1, further comprising a tube 500, wherein the tube 500 is configured to releasably connect to a fluid inlet of the endoscope.

8. The endoscope flushing assembly 100 of claim 7, wherein the tube 500 comprises a silicone rubber.

9. The endoscope flushing assembly 100 of claim 1, further comprising a fluid receptacle 600.

10. Use of the endoscope flushing assembly 100 of claim 1 to collect a fluid sample for determining the efficacy of endoscope reprocessing.

11. A method evaluating the cleanliness of an endoscope, the method comprising:
sealing an open endoscope port with a stopper 400;
securing an adapter 200 to a stand 300;
inserting the distal end of the endoscope into the adapter 200;
connecting a first end of a tube 500 to a fluid inlet of the endoscope and a second end of the tube 500 to a fluid source comprising a sample fluid;
flushing the endoscope interior with the sample fluid;
collecting the sample fluid at an outlet 220 of the adapter 200; and
analyzing the sample fluid for the presence of a contaminant.
